(19) United States
(12) Patent Application Publication
(10) Pub. No.: US 2009/0326655 A1
(43) Pub. Date: Dec. 31, 2009

(54) Implant for Fastening Ligaments or Tendons

(75) Inventors: Winfried Mayr, Modling (AT); Gobert Skrbensky, Wien (AT); Ewald Unger, Wien (AT); Roland Huber, Wien (AT)

Correspondence Address: LERNER GREENBERG STEMER LLP, P O BOX 2480, HOLLYWOOD, FL, 33022-2480, US

(73) Assignee: MEDIZINISCHE UNIVERSITAT WIEN, Wien (AT)

(21) Appl. No.: 12/375,311

(22) PCT Filed: Jul. 26, 2007
(86) PCT No.: PCT/AT07/00364

§ 371 Date: Jan. 27, 2009

(30) Foreign Application Priority Data
Jul. 27, 2006 (AT) A 1278/2006

(51) Int. Cl. A61F 2/08

(52) U.S. Cl. 623/13.13; 623/13.14

(57) ABSTRACT

An implant for fastening ligaments or tendons to a bone or bone replacement, generally referred to as a bone member. The implant has a housing, a device for fixing or attaching a ligament or tendon, and a device for being fastened to the bone member. The fixing device can be displaced relative to the fastening device. At least one motor is provided for displacing the fixing device relative to the fastening device. The motor is connected to a receiver for telemetrically triggering the motor. The implant allows the tension of the ligament or tendon and the position of the attachment of the ligament or the tendon to be post-operatively adjusted in an optimal fashion.

The invention relates to an implant for attaching ligaments or tendons to a bone or bone replacement, said implant comprising a housing, means for fixing the ligament or tendon, and comprising means for attaching said implant to the bone or bone replacement, the fixing means being arranged in an adjustable manner with respect to the attachment means, with at least one motor being provided to adjust the fixing means with respect to the attachment means, said motor being connected to a receiver for telemetric control of the motor.

Anatomic ligaments are used to flexibly connect movable parts of the bone skeleton and to restrict the movement to a functionally expedient extent. Ligaments connect bones to bones, whereas tendons connect bones to muscles. The present invention can be applied in equal measures to ligaments and tendons in both human and animal organisms.

After accidents or injuries, or even when replacing bones by prostheses, it is often necessary to attach ligaments or tendons to a bone or bone replacement with the aid of appropriate implants. This is true in equal measures for natural ligaments or tendons, and artificial ligaments or tendons, or ligament or tendon transplants. The tendons and ligaments are tightened during the operation, possibly with the aid of measurement devices, according to the wishes of the surgeon. However, after the operation, it is often found that the tension in the ligament or tendon, and/or the position of the ligament attachment site or tendon attachment site has not been optimally adjusted, and the respective joint cannot move in an optimum fashion. This makes additional surgical procedures necessary, in which the tension in the ligament or tendon is adjusted, or the ligament attachment site or tendon attachment site is repositioned. Although such surgical procedures are already in part minimally invasive, they nevertheless constitute a relatively high burden for the patient and require relatively large amounts of time and money.

Even if a joint, in particular the knee joint, is completely removed, as may be necessary after serious accidents, bone tumors or chronic inflammations, optimal reattachment of the ligaments and tendons, in particular the patellar tendon, is required. Due to the difficult skin conditions, this fixing is effected in a non-physiological manner closer to the bending axis of the knee and results in a shortening of the lever arm and hence a limitation of the functionality of the artificial joint. To correct this, a relatively complex surgical procedure is required in turn.

WO 98/18409 A1 discloses an implant for fixing a ligament to the bone, comprising a bone screw and means for fixing the end of the ligament which is connected to the screw anchored in the bone. Subsequent adjustment of the tension in the ligament is possible in a relatively complex fashion by changing the position of the bone screw or manual shortening of the ligament on the fixing means.

U.S. Pat. No. 4,708,132 A also describes a device for attaching a ligament prosthesis or tendon prosthesis to a bone, with a change of the tension in the ligament or tendon only being possible in a relatively complex and invasive fashion.

A relatively simple method for adjusting the tension is described in U.S. Pat. No. 6,558,389 B2, with the end of the ligament or tendon being connected to an element which for example has latching protrusions such that displacing the element with respect to a sleeve anchored in the bone makes it possible to change the length of, and hence the tension in, the ligament or tendon in certain steps. However, a postoperative change in the ligament tension or tendon tension requires at least a small surgical procedure.

WO 1997/36557 A1 describes an implant for attaching ligaments, by means of which fixing the ligament to the bone and applying tension in the ligament can be simplified. The fixing means can be anchored in at least two positions; however, a small surgical procedure is generally required for this. Nevertheless, telemetric adjustability is also mentioned, although no concrete embodiments are described. However, the adjustability is restricted to only the tension in the ligament fixed by the implant and this is usually insufficient for optimal adjustment.

DE 42 38 039 A1 describes an implant for increasing the length of a ligament, with the tension in the ligament being increased in steps, with the object of achieving an elongation of the ligament as a result of the directed tension. This system is not used for optimal biomechanical setting of a ligament attachment site or tendon attachment site, but for returning the length of a tendon, shortened as the result of an operation, to its original length by the stretching load in the longitudinal direction.

The object of the present invention is to provide an implant of the type mentioned above, by means of which it is possible to adjust, even postoperatively, the ligament tension or tendon tension and the position of the ligament attachment site or tendon attachment site in a manner which is as quick and simple as possible. The disadvantages of known systems should be avoided or at least reduced.

The object according to the invention is achieved by an implant as mentioned above, wherein the at least one motor is provided to adjust the fixing means with respect to the attachment means in at least one direction in order to adjust the tension and attachment site of the ligament or tendon. Hence, the implant according to the invention makes it possible to change the position of the fixing means with respect to the attachment means in order to change the tension in the ligament or tendon and to change the ligament attachment site or tendon attachment site without a surgical procedure. The adjustment of both the ligament tension or tendon tension and the ligament attachment site or tendon attachment site can be effected by a movement of the fixing means with respect to the attachment means in any direction bar the purely axial direction. Hence, the receiver which is connected to the motor or an appropriate motor control unit can receive signals through the skin, by means of which signals the at least one motor can be appropriately controlled. Thus, adjusting the ligament tension or tendon tension and the position of the ligament or tendon is possible postoperatively without a surgical procedure. In theory, it would even be feasible, in certain situations, to change the ligament tension or tendon tension for a short time only and thereafter return to the normal ligament tension or tendon tension again. For example, such an adjustment could be undertaken during competition in elite sport events. Optimal movement of the joint is achieved for the respective situation by means of optimal setting of the ligament or tendon.

In the process, it is possible that at least one motor is designed to adjust the fixing means in the longitudinal direction of the ligament or tendon. This adjustment makes optimal setting of the ligament tension or tendon tension possible.

If, as an alternative to the above, or additionally, at least one motor is designed to adjust the fixing means at an angle to the longitudinal direction of the ligament or tendon, it is also possible to adjust the position of the ligament attachment site or tendon attachment site in addition to or as an alternative to the ligament tension or tendon tension and hence the movability of the joint can be influenced. It is also possible for a plurality of motors or a motor with an appropriate transmission to be provided in order to be able to change the position of the ligament attachment site or tendon attachment site through different solid angles.

If a transmitter is provided in the implant, it is possible to transmit certain data, such as unambiguous identification or else measurement values, to the outside, where it can for example be used to assist the responsible medical practitioner with the diagnosis or therapy.

The receiver and possibly the transmitter are preferably a radiofrequency receiver or radiofrequency transmitter, respectively, in a frequency range suitable for close-field telemetry. It is likewise possible for the transmitter to combine two telemetric methods. In particular, this makes it possible for the energy and data transfer to be separated by, for example, coupling-in the energy using radiofrequency whereas the data is transmitted via an additional connection, such as an optical, magnetic or ultrasound telemetry link.

If a data memory is provided in the implant, it is possible to buffer patient-relevant data or else measurement values and these can, when necessary, be read by the treating medical practitioner, for example.

The fixing means for attaching the ligament or tendon can be a clamp in which the end of the ligament or tendon can be gripped.

In this case, it is advantageous, for a secure grip of the ligament or tendon in the clamp, if the clamp has two contoured plates for clamping the ligament or tendon.

Additionally, it is also possible for the fixing means to be an element around which the end of the ligament or tendon is looped and fixed.

In accordance with a further feature of the invention, the at least one motor and possibly further electronic components of the implant are connected to a corresponding voltage supply.

The voltage can be supplied by batteries, in particular rechargeable batteries. Advantageously, the batteries are charged from the outside by inductively coupling-in the energy such that no surgical procedure is required to charge the batteries.

The voltage can likewise be supplied directly by a circuit for inductively coupling-in energy and the at least one motor of the implant is only supplied with energy when required.

At least one motor can be a linear drive. Such motors, which operate in accordance with the piezoelectric effect for example, can be acquired with particularly small dimensions and are thus particularly suitable for the application in an implant.

Likewise, a motor can be a rotational motor with an appropriate transmission.

In this case, the transmission is preferably a self-inhibiting transmission or a transmission that can be actively inhibited so that an undesired change in the ligament fixing or tendon fixing is prevented if the supply voltage is lost. A self-inhibiting transmission blocks in the rest state but can be driven anytime without a transition, while a transmission that can be actively inhibited can be blocked in the rest state by a braking mechanism or locking mechanism and can be freed by active control measures. This can for example be implemented by means of a catch or the like.

The transmission can also be a planetary transmission.

If at least one sensor is arranged to measure the tension in the ligament or tendon, it is possible to carry out appropriate monitoring while the ligament tension or tendon tension is adjusted.

By way of example, such a sensor can be a strain gauge which is arranged on the fixing means or an element connected thereto. Strain gauges are comparatively cheap and can be produced to have very small dimensions.

The provision of a device for detecting the current intake of the at least one motor is also advantageous because the ligament tension or tendon tension can also be deduced from the current intake of the motor. Detecting the current intake of the motor can also in a simple fashion prevent overstretching of the ligament or tendon.

If a corresponding control device is provided which is connected to at least one motor and to at least one sensor and/or the detection device, then it is also possible to automatically adjust the ligament tension or tendon tension.

The means for attaching the implant to the bone or bone replacement can be appropriate bores in the housing for holding bone screws or the like. This provides a simple and efficient option for fixing the implant on the bone or bone replacement.

A further option is to design the housing to be rotationally symmetric, with a male thread provided such that the housing itself can be screwed into a bore in the bone or bone replacement and in this manner can be attached to the bone or bone replacement.

If at least one motor is arranged eccentrically in the housing in the case of a rotationally symmetric design of the housing, rotating the housing with respect to the bone or bone replacement can achieve a change in ligament attachment site or tendon attachment site within defined limits.

The housing of the implant can be made of ceramics or metal, in particular titanium.

If the housing is at least in part coated with biocompatible material, then incorporating the implant in the bone or bone replacement, or the tissue surrounding the bone, is accelerated.

The present invention will be explained in more detail on the basis of the attached drawings, in which:

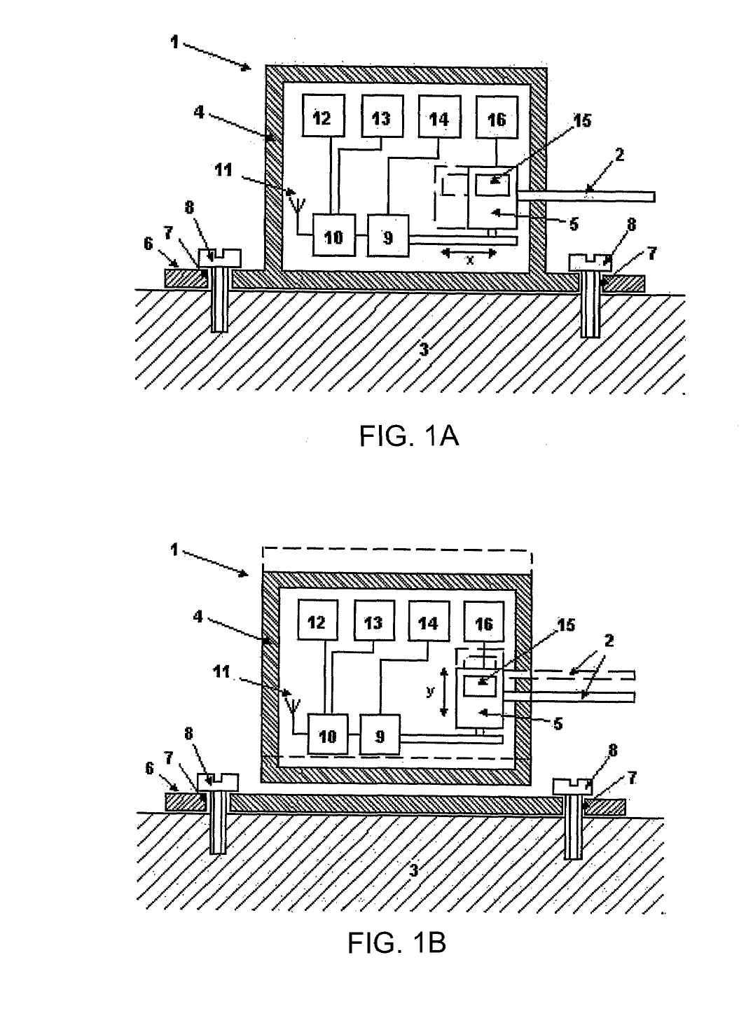

FIGS. 1a and 1b show two basic outlines of different embodiments of an implant according to the invention;

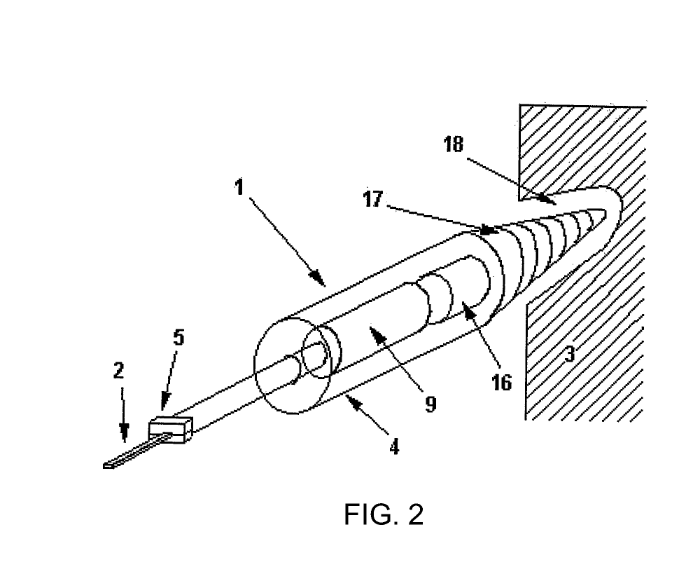

FIG. 2 shows a further embodiment of an implant with a rotationally symmetric housing;

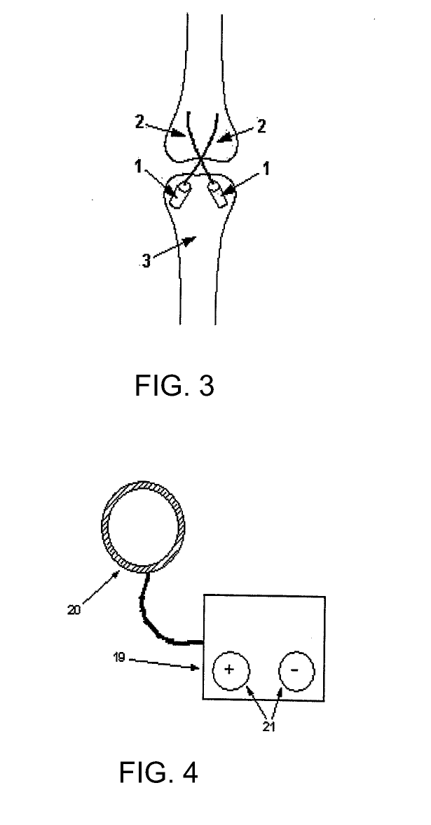

FIG. 3 shows the application of implants according to the invention for attaching ligaments or tendons to the knee joint;

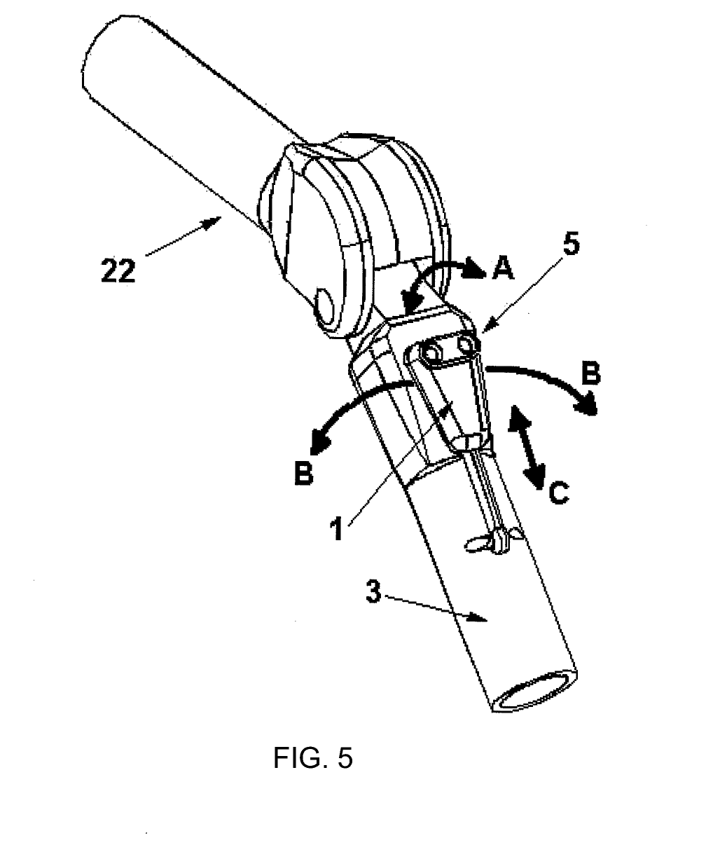

FIG. 4 shows a schematic block diagram of an appropriate drive unit; and

FIG. 5 shows an application of an implant for attaching ligaments or tendons to a knee prosthesis.

FIG. 1a shows one variant of an implant 1 according to the invention for attaching ligaments 2 or tendons to a bone 3 or bone replacement. The implant 1 comprises a housing 4 and means 5 for fixing the ligament 2 or tendon. There are a number of different designs for this fixing means 5; it could, for example, be a clamp or the like.

Furthermore, means 6 are provided for attaching the implant 1 to the bone 3 or bone replacement; these means can for example be formed by bores 7 on the housing 4, through which bores appropriate bone screws 8 or the like can be screwed into the bone 3 or bone replacement. Known designs of such implants 1 make it possible to a certain extent to adjust the fixing means 5 with respect to the attachment means 6 in order to adjust the tension in the ligament 2 or tendon.

The present invention provides for at least one motor 9 to adjust the fixing means 5 with respect to the attachment means 6 in at least one direction X, and the motor 9 is connected to a receiver 10 for the telemetric control of the motor 9. In order to obtain an adjustment both of the tension in the ligament 2 or tendon and also of the attachment site of the ligament 2 or tendon, it is necessary to adjust the fixing means 5 with respect to the attachment means 6 in a direction differing from the axial direction of the ligament 2 or tendon. Likewise, the provision of two or more motors for moving the fixing means 5 in a number of directions with respect to the attachment means 6 is possible. An appropriate signal, which is used to control the motor 9, can be fed telemetrically to the receiver 10 from the outside via a schematically indicated antenna 11. This makes it possible to readjust the tension in the ligament 2 or tendon without a surgical procedure. The motor 9 can be a linear drive or rotational motor with an appropriate transmission. Here, the transmission is preferably self-inhibiting or can be actively inhibited, with the transmission being locked in the rest state. Of course, it is also possible that a number of motors 9 are provided which make it possible to adjust the fixing means 5 with respect to the attachment means 6 or housing 4 of the implant 1 in other directions as well, as a result of which, for example, the attachment site of the ligament 2 or tendon can also be adjusted (not illustrated). In order to prevent the ligament fixing or tendon fixing from being adjusted if the voltage supply is lost, it is preferable for the transmission to be self-inhibiting.

If a transmitter 12 is provided as well, or the receiver is designed for bidirectional data interchange, it is also possible to send data from the implant 1 to the outside. The receiver 10 and possibly the transmitter 12 are preferably a radiofrequency receiver and radiofrequency transmitter, respectively. Furthermore, it is possible that a data memory 13 is provided, in which specific data about the patient or the implant 1, and also measurement data, can be saved. The motor 9 is operated by a voltage supply 14 which can be formed by batteries, in particular rechargeable batteries, or else by a circuit for inductively coupling-in energy. In the latter case, the energy required for operating the motor 9 and activating other electronic components of the implant 1 is inductively coupled into the supply voltage 14 from the outside when necessary. Such a circuit for inductively coupling-in energy (not illustrated) can also be used to recharge the batteries.

It is also advantageous if a sensor 15 is provided for measuring the tension in the ligament 2 or tendon because this can prevent overstretching the ligament 2 or tendon. Such a sensor 15 can, for example, be in the form of a strain gauge which is arranged on the fixing means 5 or an element attached thereto. In addition to, or as an alternative to, such a sensor 15, the provision of a device for detecting the current intake of the at least one motor 9 is also possible because the tension in the ligament 2 or tendon can also be deduced from the current intake. It is also possible to arrange further sensors for measuring relevant physiological parameters.

If the motor 9 and the sensor 15 and a possible device for detecting the current intake of the motor 9 are connected to a control device 16, closed-loop automated control of the tension in the ligament 2 or tendon is also possible.

The housing 4 can be made of ceramics or metal, in particular titanium, and can also at least in part be coated with biocompatible material.

FIG. 1b shows a further embodiment of an implant 1, in which the housing 4 is arranged separately from the attachment means 6 in order to adjust the housing 4 with respect to the attachment means 6, for example in the Y direction, via a motor 9 by using the fixing means 5 arranged fixedly in the housing 4. By way of example, this could make it possible to change the position of the attachment site of the ligament 2 or tendon. By using a corresponding number of motors 9, it is possible to effect a change in different directions X, Y, and hence it is possible to change both the tension in the ligament 2 or tendon and the attachment site of the ligament 2 or tendon.

FIG. 2 shows a variant of the invention, according to which the housing 4 of the implant 1 is designed to be rotationally symmetric and comprises a male thread 17 such that the housing 4 can be screwed into and hence be fixed in a bore 18 in the bone 3 or bone replacement. If the motor 9 is arranged eccentrically in the rotationally symmetric housing 4 of the implant 1, rotating the housing 4 in the bore 18 of the bone 3 or the bone replacement can achieve a change in the attachment site of the ligament 2 or tendon within certain limits.

FIG. 3 shows an application of implants 1 according to the invention in a knee joint, with two implants 1 being inserted into corresponding bores in the bone 3 and being connected to ligaments 2 which are the cruciate ligaments in the illustrated example.

A control device 19 illustrated in FIG. 4 can feed energy into the implant 1 via a transmission coil 20 and can be used to send corresponding control signals to the implants 1. The signals are received in the implant 1 and are converted into corresponding control signals for the motor 9 in the implant 1. Appropriate actuating elements 21 are provided to actuate the control device 19.

Finally, FIG. 5 shows an application of the implant 1 according to the invention in a knee prosthesis 22. Here, the implant 1 is attached to the bone replacement 3 of the knee prosthesis 22 in an adjustable manner. The ligaments or tendons (not illustrated) are connected to the implant 1 by means of an appropriate fixing means 5. The implant 1 can be moved in very different directions, as indicated by the arrows A, B and C, by means of a corresponding mechanical, hydraulic or electromechanical drive with a corresponding control. This makes it possible to telemetrically adjust the tendon tension and tendon position and makes it possible to obtain optimal movability of the artificial knee joint.

The present implant is not limited to the specified application examples; rather it can be used wherever a postoperative change in a ligament tension or tendon tension or in the position of the attachment site of the ligament or tendon is required.

1-26. (canceled)

27. An implant for attaching ligaments or tendons to a bone member, comprising:
a housing;
an attachment device for attaching said implant to the bone member;
a fixing device for fixing the ligament or tendon, said fixing device being arranged in an adjustable manner with respect to said attachment device;
at least one motor; and
a receiver;
said at least one motor being connected for adjusting said fixing device relative to said attachment device in at least one direction for adjusting tension and a site of attachment of the ligament or tendon and to said receiver for telemetrically controlling said motor.

28. The implant according to claim 27, wherein said at least one motor is arranged to adjust said fixing device in a longitudinal direction of the ligament or tendon.

29. The implant according to claim 27, wherein said at least one motor is arranged to adjust said fixing device at an angle relative to a longitudinal direction of the ligament or tendon.

30. The implant according to claim 27, further comprising a transmitter.

31. The implant according to claim 30, wherein at least one of said receiver and said transmitter is radiofrequency responsive.

32. The implant according to claim 27, including a data memory.

33. The implant according to claim 27, wherein said fixing device is a clamp.

34. The implant according to claim 33, wherein said clamp comprises two contoured plates for clamping the ligament or tendon.

35. The implant according to claim 27, wherein an end of the ligament or tendon is disposed around said fixing device.

36. The implant according to claim 27, including a voltage supply connected to said at least one motor.

37. The implant according to claim 36, wherein said voltage supply is a battery.

38. The implant according to claim 36, said voltage supply comprises an inductively coupled circuit.

39. The implant according to claim 27, wherein said at least one motor is a linear drive.

40. The implant according to claim 27, wherein said at least one motor is a rotational motor having a transmission.

41. The implant according to claim 40, wherein said transmission is one of a self-inhibiting or actively inhibited transmission.

42. The implant according to in claim 40, wherein said transmission is a planetary transmission.

43. The implant according to in claim 27, including at least one sensor arranged to measure tension in the ligament or tendon.

44. The implant according to in claim 43, wherein said at least one sensor is a strain gauge arranged on one of said fixing device or an element connected thereto.

45. The implant according to claim 27, including a device to detect a current intake of said at least one motor.

46. The implant according to claim 43, including a control device connected to said at least one motor and at least one of said at least one sensor or a detection device to detect current intake of said at least one motor.

47. The implant according to claim 27, wherein said attachment device comprises bores on said housing for holding fastening members.

48. The implant according to claim 27, wherein said housing is constructed to be rotationally symmetrical and includes a male thread to enable said housing to be screwed into a bore in the bone member.

49. The implant according to claim 48, wherein said at least one motor is disposed eccentrically in said housing.

50. The implant according to claim 27, wherein said housing is ceramic.

51. The implant according to claim 27, wherein said housing is a metal housing.

52. The implant according to claim 27, wherein said housing is at least partially coated with a biocompatible material.

53. The implant according to claim 27, wherein the bone member is a bone or bone replacement.
